(19) United States
(12) Patent Application Publication
(10) Pub. No.: US 2002/0120176 A1
(43) Pub. Date: Aug. 29, 2002

(54) Blanket with recordable heart beat

(76) Inventor: Peter Coviello, Chatham, NJ (US)

Correspondence Address: PETER COVIELLO, 21 Spring Street, Chatham, NJ, 07928, US

(21) Appl. No.: 09/794,030

(22) Filed: Feb. 28, 2001

(51) Int. Cl. A61M 21/00

(52) U.S. Cl. 600/28

(57) ABSTRACT

A blanket which incorporates apparatus for recording and playing back sounds of a beating heart, for soothing babies. The blanket has a pocket which receives a sound generator including a microprocessor chip, an e-prom memory chip, a piezo speaker, a solid state pick up transducer, a miniature battery cell, and preferably three control pushbuttons. Two of the pushbuttons must be pressed simultaneously to activate the recording function. The shut off function is similarly controlled, with different pushbuttons. Optionally, the pick up transducer is connected to the sound generator by a cable having a manually removable connector. In another option, a radio frequency transmitter is operably connected to the sound generator to enable operation as an audible monitor when the recording function is also activated.

### BACKGROUND OF THE INVENTION

1. Field of the Invention

The present invention relates to blankets, and more particularly to modification of a blanket to incorporate a sound source for reproducing the sound of a heart beat.

2. Description of the Prior Art

It has long been established that people respond favorably to the sound of human heart beat. This is particularly true of infants and babies who are being readied for sleep. It is obviously not feasible for a parent or other care giver to remain in proximity to a child in order to provide the effect of the sound of heart beat. Artificial reproduction of heart beat has been proposed in the prior art to provide the benefits of the sound of heart beat while not requiring physical presence of another person. Children being put to sleep are frequently covered with blankets for comfort and warmth. It would be desirable to combine the benefits of a blanket with a device which provides audible heart beat stimulus.

U.S. Pat. No. 5,205,811, issued to Belinda J. Fornarelli on Apr. 27, 1993, describes a baby blanket having a heart beat simulator. However, unlike the present invention, the heart beat simulator is encased in synthetic foam. Also, the simulator does not teach recording and playback of actual sound. Beyond the foregoing, circuitry and other features of the novel device are not shown in Fornarelli.

U.S. Pat. Nos. 4,124,022, issued to Sam Gross on Nov. 7, 1978, 5,063,912, issued to John S. Hughes on Nov. 12, 1991, and 6,004,259, issued to Shawky Sedaros on Dec. 21, 1999, all show devices which reproduce or approximate the sound of a beating heart for calming babies and inducing sleep. None of these devices is associated with a blanket.

U.S. Pat. No. 3,994,282, issued to Camille Moulet on Nov. 30, 1976, illustrates apparatus for reproducing simulated sound of human heart beat. This device generates simulated sound rather than recording actual sound, and furthermore is not associated with a blanket.

None of the above inventions and patents, taken either singly or in combination, is seen to describe the instant invention as claimed.

### SUMMARY OF THE INVENTION

The present invention provides a blanket associated with a recording and playback device, or sound generator, for recording sounds of heart beat and audibly reproducing the recorded sounds for purposes of comforting and relaxing a small child. The sound generator utilizes electronic circuitry, and is concealed and contained within the blanket. In alternative embodiments, the blanket is hollow and opens at one end to receive the sound generator, or alternatively has an external pocket for receiving the sound generator. The sound generator is small and flat, being similar in configuration to a credit card, and thus is readily carried unobtrusively within the blanket.

Electronic circuitry enables digital recording and play back in a continuous loop. This feature minimizes the length of time that heart beat must be recorded in order to be able to play back heart beat sounds for an extended period of time. The recorder may be either integral with the sound generator or separate therefrom. If separate, it has a flexible cable and is plugged into the sound generator. A speaker is contained within the sound generator. A miniature central processing unit and memory chip are connected to the recorder and speaker, and are powered by a miniature battery cell. Three control buttons are provided. Two of these buttons must be operated simultaneously to enter commands to cease replaying sound and to establish a new recording. This feature prevents unintended shut off and rerecording which might occur as a child rolls over the sound generator.

Accordingly, it is one object of the invention to provide a blanket having capability of recording and playing the sound of a heart beating.

It is another object of the invention that sound reproducing apparatus be carried unobtrusively with the blanket.

It is a further object of the invention to enable long term replaying of heart beat sounds while requiring a relatively short period of recording.

It is an object of the invention to provide improved elements and arrangements thereof in an apparatus for the purposes described which is inexpensive, dependable and fully effective in accomplishing its intended purposes.

These and other objects of the present invention will become readily apparent upon further review of the following specification and drawings.

### BRIEF DESCRIPTION OF THE DRAWINGS

Various other objects, features, and attendant advantages of the present invention will become more fully appreciated as the same becomes better understood when considered in conjunction with the accompanying drawings, in which like reference characters designate the same or similar parts throughout the several views, and wherein:

FIG. 1 is a top plan view of one embodiment of a blanket according to the invention.

FIG. 2 is a front perspective detail view of a sound generating component shown at the upper right of FIG. 1.

FIG. 3 is a schematic representation of functional components of the sound generating component of FIG. 2.

FIG. 4 is a front perspective detail view of an alternative embodiment of the sound generating component of FIG. 2.

### DETAILED DESCRIPTION OF THE PREFERRED EMBODIMENTS

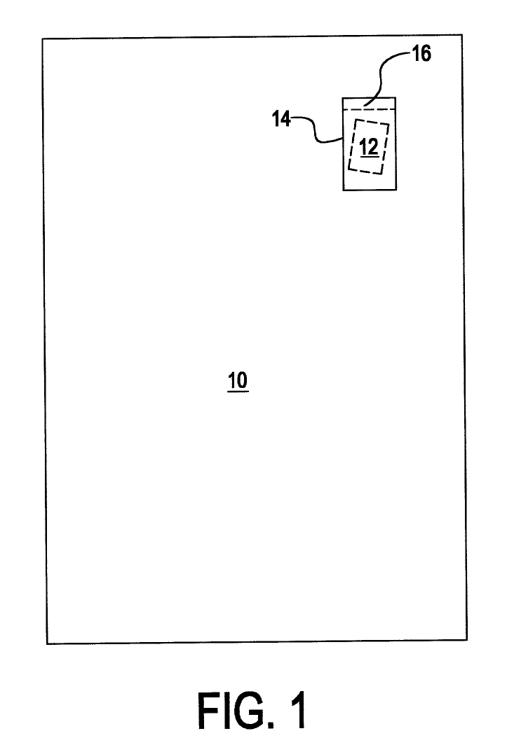

Turning now to FIG. 1 of the drawings, there is shown a blanket 10 having an associated sound generator 12 secured thereto in an enclosed compartment. The compartment is formed by a pocket 14 which is dimensioned and configured to receive sound generator 12. Blanket 10 may be of any suitable construction, for example comprising a panel of cotton or fleece, with or without a border of other material. Pocket 14 is formed by fixing a rectangular external section of cloth to the panel of blanket 10, such as by stitching along three sides of the periphery of the rectangle, and leaving the fourth side open. The open fourth side is provided with a manually openable closure such as complementing patches of hook and loop material, indicated representatively at 16, to maintain sound generator 12 within pocket 14. Hook and loop material of one polarity or type is adhered to the panel of blanket 10, and hook and loop material of the other polarity or type is adhered to the external section of cloth. Pocket 14 may therefore be opened by pulling the external section of cloth away from the panel of blanket 10, for inserting and retrieving sound generator 12.

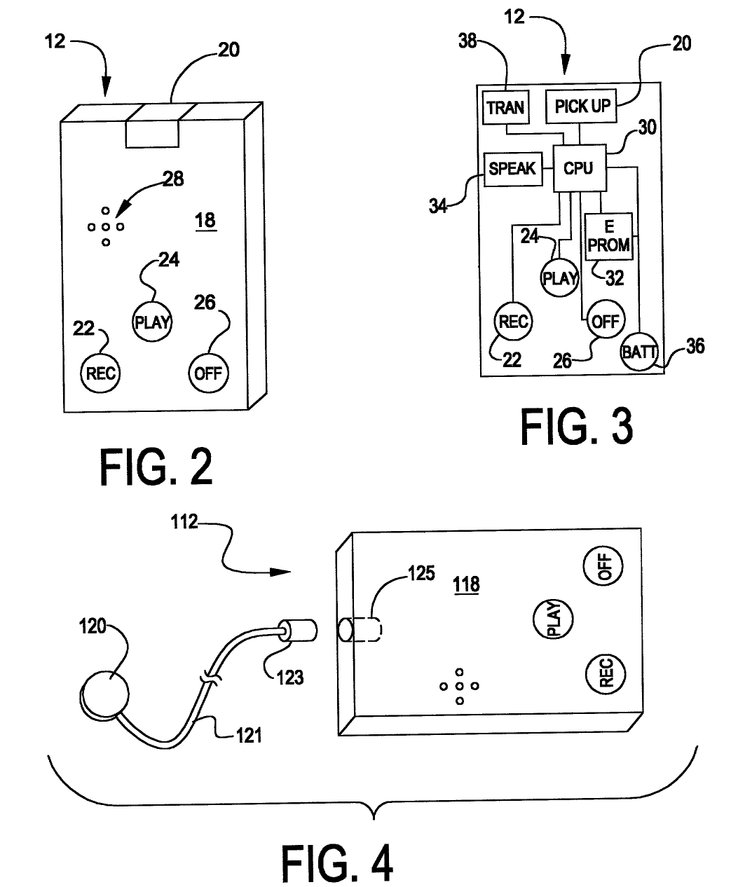

FIG. 2 shows one embodiment of sound generator 12, the function of which is to record and play back sounds of heart beat on demand. Sound generator 12 incorporates small components enclosed within a housing 18. Housing 18 may have dimensions and proportions roughly of those of a plastic card such as a credit or debit card (not shown). That is, housing 18 may be as small as three inches in length and two inches in width, with thickness on the order of one sixteenth or one eighth of an inch. Precise size and proportions of housing 18 are not critical. Similarity to those of a credit card are described for illustration only. Obviously, size and proportions may be varied to suit. Housing 18 may be formed from a suitable plastic material which is sufficiently rigid to hold operable components in a fixed position within housing 18 and to protect the operable components from damage when subjected to normal use. Normal use contemplates the maximum force which can be generated by a young child lying on housing 18.

Some of the operable components of sound generator 12 are externally exposed. Exposed components include a digital sound pick up device such as solid state pick up transducer 20, and three control pushbuttons 22, 24, 26, the function of which will be described hereinafter. Housing 18 is perforated, as indicated generally at 28, to enable sound generated by a sound synthesizer to project from housing 18.

FIG. 3 shows the operable components of sound generator 12 and their interrelationships. These components include a microprocessor or central processing unit such as micro chip 30, a memory chip such as e-prom chip 32, and a battery cell 36, in addition to components visible or accessible from the exterior of housing 18. Battery cell 36, which may be a miniature battery cell of a type provided to power small appliances such as wrist watches, is operably connected to power consuming components. Solid state pick up transducer 20, the sound synthesizer, which is preferably a piezo speaker 34, and control pushbuttons 22, 24, 26 are operably connected to micro chip 30 selectively to cause said sound generator to record new sound, play recorded sounds, and to cease playing recorded sounds.

Control pushbuttons 22, 24, 26 operate in the following manner. Recordings are made by operating pushbutton 22, designated REC as an abbreviation of “record”, in combination with pushbutton 24, designated PLAY. PLAY pushbutton 24 is depressed to play back recorded sounds. Pushbutton 26, designated OFF, discontinues play back when depressed in combination with PLAY pushbutton 24.

It is important to prevent unintended shutting off of operation of sound generator 12, as might occur by incidental contact by a baby who in the course of moving could possibly bring his or her weight to bear on a pushbutton 22, 24, or 26. Similarly, it is important to prevent unintended recording operation by similar incidental contact with any of pushbuttons 22, 24, or 26. Therefore, the three pushbuttons 22, 24, 26 are provided in spaced apart location on housing 18. Also, simultaneous operation of two predetermined ones of the pushbuttons 22, 24, 26 are required to bring about recording and shutting off of replay of recorded sound functions.

A miniature low powered radio frequency transmitter 38 is optionally provided to enable sound generator 12 to function as an audible monitor capable of picking up sounds from the vicinity of blanket 10 and transmitting the sounds as signals to a remote receiver (not shown) which is compatible with said transmitter and which may of any known type suitable for use in monitoring babies. Transmitter 38 is operably connected to micro chip 30 to transmit sound signals to the remote receiver, and to battery cell 36. To operate in the monitoring mode, the recording function is activated by operating the sound pick up device, while transmitter 38 is simultaneously operated. To operate transmitter 38, an additional control pushbutton (not shown) may be provided, or alternatively a previously unused permutation or scheme of simultaneous operation of pushbuttons 22, 24, 26 may be utilized.

FIG. 4 shows an alternative embodiment of the invention wherein the sound pick up device is manually removable from housing 118 of sound generator 112. Solid state pick up transducer 120 has a flexible communication cable 121 which terminates in a first connector terminal 123. A second connector which is manually attachable to and detachable from the first connector, such as socket 125, is provided in housing 118 for receiving terminal 123. Sound generator 112 is functionally similar to sound generator 12 of FIG. 3, having comparable components which are not specifically described with reference to the embodiment of FIG. 4. The only difference between the embodiments of FIGS. 2 and 4 is the feature wherein the sound pick up device is selectively attachable to and removable from housing 118.

The present invention is susceptible to variations and modifications which may be introduced thereto without departing from the inventive concept. For example, in place of a pocket which is considerably smaller than the principal fabric panel of the blanket, the blanket itself may form one large pocket. In another example, the sound synthesizer may be exposed to view rather than contained within the housing of the sound generator.

It is to be understood that the present invention is not limited to the embodiments described above, but encompasses any and all embodiments within the scope of the following claims.

I claim:

1. A blanket having an associated sound generator disposed to record and play back sounds of heart beat, comprising:
a sound generator having a digital sound pick up device, a sound synthesizer, a microprocessor, and a memory chip all operably interconnected to one another, and manual controls operably connected to said microprocessor selectively to cause said sound generator to record new sound, play recorded sounds, and to cease playing recorded sounds; and
a blanket having an enclosed compartment dimensioned and configured to receiving said sound generator, wherein said compartment has a manually openable closure disposed to maintain said sound generator within said compartment.

2. The blanket according to claim 1, wherein said sound generator has a housing disposed to enclose and to hold said sound pick up device, said sound synthesizer, said microprocessor, and said memory chip in a fixed position within said housing.

3. The blanket according to claim 1, wherein said sound generator has an enclosure bearing a first connector, and said sound pick up device has a second connector which is manually attachable to and detachable from said first connector so that said sound pick up device is selectively manually attachable to and detachable from said housing of said sound generator.

4. The blanket according to claim 1, wherein said microprocessor comprises a microchip, and said memory chip is an e-prom memory chip.

5. The blanket according to claim 1, wherein said sound pick up device is a solid state pick up transducer.

6. The blanket according to claim 1, wherein said sound synthesizer is a piezo speaker.

7. The blanket according to claim 1, wherein said manual controls comprise at least three pushbuttons spaced apart from one another, arranged so that two predetermined ones of said pushbuttons must be pressed simultaneously to initiate recording.

8. The blanket according to claim 1, wherein said manual controls comprise at least three pushbuttons spaced apart from one another, arranged so that two predetermined ones of said pushbuttons must be pressed simultaneously to stop replay of recorded sound.

9. The blanket according to claim 1, wherein said enclosed compartment comprises an open pocket fixed to said blanket.

10. The blanket according to claim 1, further including a radio frequency transmitter operably connected to said microprocessor to transmit sound while said sound pick up device is operating, so that said sound generator serves as a monitor capable of picking up sounds from the vicinity of said blanket and transmitting the sounds as signals to a remote receiver which is compatible with said transmitter.

11. The blanket according to claim 1, further including a battery cell operably connected to said sound pick up device, said sound synthesizer, said microprocessor, said memory chip, and said manual controls.
